(19) United States
(12) Patent Application Publication
(10) Pub. No.: US 2025/0277115 A1
(43) Pub. Date: Sep. 4, 2025

(54) PIGMENT DISPERSION FOR AQUEOUS COSMETIC COMPOSITIONS, AND AQUEOUS COSMETIC COMPOSITION USING THE SAME

(71) Applicant: MITSUBISHI PENCIL COMPANY, LIMITED, Tokyo (JP)

(72) Inventor: Yoshimune FURUKAWA, Tokyo (JP)

(73) Assignee: MITSUBISHI PENCIL COMPANY, LIMITED, Tokyo (JP)

(21) Appl. No.: 19/061,056

(22) Filed: Feb. 24, 2025

(30) Foreign Application Priority Data
Feb. 29, 2024 (JP) 2024-030820
Feb. 14, 2025 (JP) 2025-022411

(51) Int. Cl. C09B 67/20; A61K 8/06; A61K 8/46; A61K 8/81; A61Q 1/10; C09B 45/22

(52) U.S. Cl. CPC C09B 67/006; A61K 8/06; A61K 8/466; A61K 8/8141; A61Q 1/10; C09B 45/22; A61K 2800/43; A61K 2800/5422; A61K 2800/5424; A61K 2800/58; A61K 2800/594; A61K 2800/872; A61K 2800/95

(57) ABSTRACT

A pigment dispersion for aqueous cosmetic compositions and an aqueous cosmetic composition, the pigment dispersion comprising, at least an aluminum lake such as Red No. 40; at least one dispersant selected from polyorganic acids and salts thereof, and nonionic surfactants; and water and a water-soluble organic solvent.

### FIELD

The present invention relates to: a pigment dispersion for aqueous cosmetic compositions, which is excellent in the dispersibility of aluminum lake pigments such as Red No. 40 and the stability with time; and an aqueous cosmetic composition using the same.

### BACKGROUND

Conventionally, a large number of materials containing a dye or a pigment have been used as coloring materials of makeup cosmetics such as eyeliners and eyebrow cosmetics that are applied to keratin surfaces of the skin and the like. However, dyes are being avoided and not used in many cases since they can leave a color after use by causing staining on keratin surfaces of the skin and the like. The present applicant has realized makeup cosmetics such as eyeliners and eyebrow cosmetics that have a low viscosity of 15 mPa·s or less at a shear rate of 192 s⁻¹, by adjusting an iron oxide pigment such as red iron oxide to have an extremely small particle size of 30 nm to 100 nm and using an anionic polymer dispersant and an acid having a molecular weight of 300 or less (PTL 1).

However, these makeup cosmetics have a limitation on the color development due to the use of the pigment, and there are problems in terms of, for example, toning and color reproducibility.

On the other hand, dye-containing liquid cosmetics and the like exhibit good color development and do not have a problem in toning and hue reproduction; however, as described above, they have problems in not only the staining property on keratin surfaces but also the light resistance and the like. Therefore, a lake pigment such as Red No. 40, in which a dye is converted into a salt by a reaction with a metal such as aluminum and thereby insolubilized in water, is used (PTL 2).

In this literature, however, the pigment is preferably in the form of large particles of up to 500 μm in size and, with regard to the characteristics of a cosmetic, an emulsion-type cosmetic having an extremely high viscosity (27,000 cPa to 434,000 cPa) is disclosed. It is extremely difficult to incorporate such a cosmetic into a so-called pen-type cosmetic product used as an eyeliner for drawing fine lines, and the ease of handling during the product thereof is poor.

Similarly, a cosmetic in which an organic pigment such as aluminum lake is blended into a composition having a high viscosity of 1.5 Pas to 3.0 Pa·s preferably at a shear rate of 100 s⁻¹ has been disclosed (PTL 3).

The cosmetic disclosed in this literature has a very high viscosity, although it is lower than that of the cosmetic of PTL 2. Nevertheless, it is said difficult to uniformly and smoothly apply this cosmetic.

Further, it has been disclosed to add an organic pigment such as an aluminum lake to a stick-form, or press-molded powder or loose-powder cosmetic (PTL 4).

Even in this form, good performance is obtained in terms of vivid color development and color reproducibility; however, it is said to be difficult to uniformly and smoothly apply the cosmetic in a sharp line.

### CITATION LIST

### Patent Literature

[PTL 1] Japanese Unexamined Patent Publication (Kokai) No. 2015-164912 (Claims, Examples, Abstract, etc.)
[PTL 2] Japanese Unexamined Patent Application Publication (Translation of PCT Application) No. 2012-504570 (Claims, paragraphs [0050], [0251], etc.)
[PTL 3] Japanese Unexamined Patent Publication (Kokai) No. 2023-88285 (Claims, paragraphs [0037], [0038], [0039], etc.)
[PTL 4] Japanese Patent No. 7287458 (Claims, [0045], [0055], Examples, etc.)

### SUMMARY

### Technical Problem

In view of the above, the present invention is aimed at solving the above-described conventional problems and the like, and an object of the present invention is to provide: a pigment dispersion for aqueous cosmetic compositions, which is excellent in the color development of pigments such as Red No. 40, the color reproducibility, the applicability, the dispersibility of aluminum lake pigments, and the stability with time; and an aqueous liquid composition using the same, which is suitable for eyeliners, eyebrow cosmetics, and the like.

### Solution to Problem

The present inventor intensively studied the above-described conventional problems and the like, and consequently developed: a pigment dispersion for aqueous cosmetic compositions, which is characterized by containing at least an aluminum lake, a dispersant, and water; and an aqueous cosmetic composition, thereby completing the present invention. Further, the present inventor discovered that, by using a composition that contains at least an aluminum lake at a ratio of 0.00025 to 30.00 mass %, 0.05 to 15.00 mass % of a dispersant, and at least 2.50 to 30.00 mass % of a film-forming agent, the above-described target pigment dispersion for aqueous cosmetic compositions and an aqueous cosmetic composition using the same, which is suitable for an eyeliner composition, an eyebrow cosmetic, or the like, can be obtained, thereby completing the present invention.

The above-described dispersant is preferably selected from polyorganic acids and salts thereof, and nonionic surfactants.

The above-described aqueous cosmetic composition preferably has a viscosity of 1.5 mPa·s to 20.0 mPa·s at 25° C. and a shear rate of 192 s⁻¹.

### Advantageous Effects of Invention

According to the present invention, the following are provided: a pigment dispersion for aqueous cosmetic compositions, which is excellent in the dispersibility and the stability with time of aluminum lakes that are pigments, such as Red No. 40; and an aqueous cosmetic composition using the same, which is excellent in the color development, the color reproducibility, and the applicability, and suitable for eyeliners, eyebrow cosmetics, and the like.

### BRIEF DESCRIPTION OF DRAWINGS

FIG. 1 is a longitudinal cross-sectional view that illustrates one exemplary embodiment of the rotary feeding-type liquid cosmetic applicator A of the present invention in a pre-use state.

FIG. 2 is a partial cross-sectional view that illustrates one exemplary embodiment of the collector-type direct-feed liquid cosmetic applicator B of the present invention.

FIG. 3 is a partial cross-sectional view that illustrates one exemplary embodiment of the inner cotton-type liquid cosmetic applicator C of the present invention.

FIG. 4A illustrates a front view of one exemplary embodiment of the knocking-type liquid cosmetic applicator D of the present invention in a pre-use state.

FIG. 4B illustrates a cross-sectional view of one exemplary embodiment of the knocking-type liquid cosmetic applicator D of the present invention in a pre-use state.

### DESCRIPTION OF EMBODIMENTS

Embodiments of the present invention will now be described in detail.

The aqueous pigment dispersion for aqueous cosmetic compositions according to the present invention is characterized by containing, at least: an aluminum lake; at least one dispersant selected from polyorganic acids and salts thereof, and nonionic surfactants; and water.

The aqueous cosmetic composition according to the present invention, which is suitable for an eyeliner composition, an eyebrow cosmetic, or the like, is characterized by containing, at least: an aluminum lake at a ratio of 0.00025 to 30.00 mass %; 0.05 to 15.00 mass % of a dispersant selected from polyorganic acids and salts thereof, and nonionic surfactants; and at least 2.50 to 30.00 mass % of a film-forming agent.

As described above, the aluminum lake pigment used in the present invention is a coloring material generally used in cosmetic products, and examples thereof include Red No. 40, Blue No. 1, and Yellow No. 4.

For example, an aqueous dispersion of Red No. 40 requires a long dispersion time, and has problems of low productivity, poor stability with time, and the like; however, these problems are solved by allowing the dispersion to have the formulation characteristics of the present invention.

From the standpoint of post-dispersion stability and convenience in the production of a cosmetic, it is desired that the content of Red No. 40, Blue No. 1, or Yellow No. 4 be preferably 1 to 32 mass %, more preferably 2 to 25 mass %, with respect to a total amount of the pigment dispersion for aqueous cosmetic compositions.

By controlling the content of Red No. 40, Blue No. 1, or Yellow No. 4 to be 1 mass % or more, not only excellent productivity is attained, but also excellent coloring property is obtained when the pigment dispersion is added to a cosmetic, while by controlling the content to be 32 mass % or less, the dispersibility and the stability with time are further improved.

In the present invention, water is mainly used as a dispersion medium and a solvent, and a water-soluble organic solvent can be further used. The water-soluble organic solvent that can be used is used as a dispersion medium of the pigment dispersion for aqueous cosmetic compositions and as a solvent of a cosmetic, and examples thereof include lower alcohols having 5 or less carbon atoms. Specifically, the water-soluble organic solvent may be, for example, at least one (one or a mixture of two or more) of methyl alcohol (methanol), ethyl alcohol (ethanol), n-propyl alcohol, isopropyl alcohol, n-butyl alcohol, sec-butyl alcohol, tert-butyl alcohol, isobutyl alcohol, pentyl alcohol, ethylene glycol, propylene glycol, butylene glycol, and the like.

Particularly, from the standpoint of safety, ease of handling, and the like, it is desirable to use ethyl alcohol (ethanol).

From the standpoint of the dissolved stability of the below-described dispersant as well as the standpoint of the stability of pigment dispersion, particularly the low-temperature stability, the content of the water-soluble organic solvent to be used is desirably 1.0 to 15.0 mass % with respect to a total amount of the pigment dispersion for aqueous cosmetic compositions. It is desired that the content of the water-soluble organic solvent together with water be more preferably 50.0 to 90.0 mass %, still more preferably 60.0 to 80.0 mass %.

By controlling the added amount of the water-soluble organic solvent to be 1.0 mass % or more, not only an effect of preventing the solvent from freezing at a low temperature can be exerted, but also an antiseptic effect is exerted although slightly. When the added amount is controlled to be 15.0 mass % or less, neither an adverse effect on the below-described film-forming agent nor irritation to the skin during use is observed. By controlling the content of water and the water-soluble organic solvent to be 50.0 mass % or more, a reduction in viscosity can be achieved and an antiseptic effect is exerted although slightly, while by controlling the content to be 90.0% mass or less, the dissolved stability of the below-described dispersant is further improved.

The dispersant used in the present invention is a component that improves the dispersibility of an aluminum lake such as Red No. 40 and the stability with time of the aluminum lake in a dispersion.

The dispersant, which is a polyorganic acid and a salt thereof, a nonionic water-soluble polymer, or a nonionic surfactant, contains a hydrophilic group in the molecule and is dissolved in water and a water-soluble solvent because of the hydrophilic group. Particles of an aluminum lake pigment such as Red No. 40 are obtained by mixing a tar dye and an aluminum salt that are soluble in water, and laking; therefore, it is believed that the particles of the aluminum lake are likely to form hydrogen bonds with the hydrophilic group of the above-described dispersant, and the hydrogen bonds exist in such a manner to envelop the particles of the aluminum lake even in water and the water-soluble solvent.

In the invention, by performing a dispersion process in water and a water-soluble solvent in the presence of a polyorganic acid and a salt thereof, a nonionic water-soluble polymer, or a nonionic surfactant, the dispersibility of an aluminum lake such as Red No. 40 and the stability with time of the aluminum lake in a dispersion are improved.

Examples of polyorganic acids and salts thereof that can be used include: polylactic acid and salts thereof; polyphosphoric acid and salts thereof; polyacrylic acid and salts thereof; and polyaspartic acid and salts thereof. Examples of the polyorganic acid salt to be used include salts of the above-described polylactic acid, polyphosphoric acid, polyacrylic acid, and polyaspartic acid, and the polyorganic acid salt may be any salt that contains at least one or more of the above-exemplified salts. Examples of these types of salts include alkali metal salts, alkaline earth metal salts, ammonium salts, and alkanolamine salts, among which sodium salts, potassium salts, and lithium salts are particularly preferred. Among the above-exemplified polyorganic acids and salts thereof, particularly polyacrylic acid and salts thereof as well as polyaspartic acid and salts thereof have excellent dispersion stability, and sodium polyacrylate and sodium polyaspartate are especially desirable. Examples of commercially available products of polyacrylic acid include POLYACRYLIC ACID 5,000 (manufactured by FUJIFILM Wako Pure Chemical Corporation) and AQUALIC H (manufactured by Nippon Shokubai Co., Ltd.).

Examples of nonionic water-soluble polymers and acrylate copolymers that can be used include AMPHOMER HC (manufactured by Nouryon Japan K.K.), LUVIMER 100P (alkyl acrylate copolymer, manufactured by BASF Ltd.), and ACULYN 33A Rheology Modifier (manufactured by Dow Chemical Japan, Ltd./Dow Toray Co., Ltd.).

Examples of nonionic surfactants that can be used include NIKKOL BB30 (behenyl polyoxyethylene 30-mol adduct) and NIKKOL BC-20 (cetyl polyoxyethylene 20-mol adduct) (both of which are manufactured by Nikko Chemicals Co., Ltd.).

From the standpoint of improving the dispersibility of an aluminum lake such as Red No. 40 and the stability with time of the aluminum lake in a dispersion, the content of the dispersant to be used is preferably 0.05 to 17.00 mass % with respect to a total amount of the pigment dispersion for aqueous cosmetic compositions, preferably in terms of solid content.

By controlling the content of the dispersant to be 0.05 mass % or more, not only the dispersed state of an aluminum lake such as Red No. 40 is stabilized, but also the adhesion of a pigment and the like when blended into a cosmetic is improved. Meanwhile, by controlling the content of the dispersant to be 17.00 mass % or less, 16.50 mass % or less, 16.00 mass % or less, 15.50 mass % or less, or 15.00 mass % or less, an increase in viscosity is inhibited, so that the convenience in the production of a cosmetic is further improved.

In the present invention, it is desired that a blending ratio of the above-described aluminum lake to be used, such as Red No. 40, and the dispersant (aluminum lake such as Red No. 40:dispersant) be more preferably 2:1 to 100:1 in terms of mass ratio.

By setting this ratio at 2:1 to 100:1, a pigment dispersion for aqueous cosmetic compositions which can satisfy the dispersibility and the stability with time of the aluminum lake such as Red No. 40 both at higher levels is obtained.

As water serving as a solvent in the present invention, for example, distilled water, ion-exchanged water, purified water, pure water, or ultrapure water can be used. A ratio of this water with respect to all solvents (water+water-soluble organic solvent) is required to be 30 to 95 mass % (0.30 to 0.95), and it is preferably 60 to 95 mass %.

When the ratio of the water with respect to all solvents is lower than 30 mass % (0.30), the stability with time is poor, and the pigment cannot be dispersed, whereas when this ratio is higher than 95 mass % (0.95), solvents other than the water that are originally contained in the respective components make it impossible to produce a pigment dispersion.

The pigment dispersion for aqueous cosmetic compositions according to the present invention that is configured in the above-described manner can solve the problems of, for example, long dispersion time and poor stability with time of aluminum lakes that are pigments, such as Red No. 40, in the dispersion, and provides a pigment dispersion for aqueous cosmetic compositions that is excellent in not only the dispersibility of aluminum lakes such as Red No. 40 but also the stability with time.

This pigment dispersion for aqueous cosmetic compositions is suitably used in the cosmetic applications where an aluminum lake such as Red No. 40 is used, and examples of preferred applications include skin care cosmetics, scalp hair cosmetics, antiperspirant cosmetics, makeup cosmetics, UV protection cosmetics, and nail cosmetics, for example: basic skin care cosmetics, such as milky lotion, creams, lotion, sunscreen agents, suntan agents, anti-acne cosmetics, and essences; makeup cosmetics, such as foundations, eye shadows, eyeliners, eyebrow cosmetics, mascaras, blushers, nail colors, treatment nails, various gel nails, and lipsticks; rinses; conditioners; hair colors; hair setting agents; hair restorers; deodorants; and fragrances. Further, the form of a product is not particularly limited and, since the pigment dispersion for aqueous cosmetic compositions is a dispersion liquid (aqueous), it can be applied to aqueous products in the form of a liquid, an emulsion, a cream, a paste, a gel, a mousse, a spray, or the like.

Particularly, the pigment dispersion for aqueous cosmetic compositions according to the present invention are, because of its dispersion characteristics and the like, preferably used in aqueous cosmetic compositions, such as eyeliners, eyebrow cosmetics, mascaras, and water-based nail colors.

The pigment dispersion for aqueous cosmetic compositions according to the present invention can be prepared by mixing the above-described aluminum lake such as Red No. 40 and dispersant with a film-forming agent, water, a water-soluble organic solvent, and other components under suitable dispersion conditions using, for example, a disperser such as a homomixer, a sand mill, an ultrasonic homogenizer, or a high-pressure homogenizer, and the average particle size of the aluminum lake pigment such as Red No. 40 in the resulting cosmetic (after dispersion) can be adjusted to be 150 nm to 350 nm. The average particle size may exceed 350 nm as long as a stable dispersed system can be obtained.

The cosmetic according to the present invention contains the above-described water-soluble organic solvent, aluminum lake such as Red No. 40, dispersant, water, and water-soluble organic solvent and, from the standpoint of further improving the dispersibility and the dissolved stability of each component, if necessary, a pH modifier, a surfactant, a viscosity modifier, a chelating agent, and the like can be used as appropriate.

Further, the pigment dispersion for aqueous cosmetic compositions according to the present invention can be produced through the steps of blending components, such as the above-described water-soluble organic solvent, aluminum lake such as Red No. 40, dispersant, and water, in the above-described respective content ranges, uniformly stirring and mixing the resultant, and then dispersing the components using a homomixer or the like.

For example, an aqueous cosmetic composition can be prepared by mixing with stirring an aluminum lake such as Red No. 40, the above-described various additives, and a solvent such as water to homogeneity using a general-purpose disperser or the like, and subsequently further mixing the resultant to homogeneity using a disperser or the like.

As a specific embodiment of the aqueous cosmetic composition according to the present invention, the use of the aqueous cosmetic composition in an eyeliner will now be described. Examples of an eyeliner composition include one which contains at least: the above-described pigment dispersion for aqueous cosmetic compositions that contains an aluminum lake such as Red No. 40; a general-purpose eyeliner component, such as a film-forming agent; and, if necessary, a thickener, a pH modifier, water, and a water-soluble organic solvent such as a lower alcohol. Depending on the color variation of the eyeliner composition, if necessary, the eyeliner composition may further contain a colorant other than the aluminum lake such as Red No. 40.

From the standpoint of effects, solubility, storage stability, and the like, it is desired that the content of the pigment dispersion for aqueous cosmetic compositions that contains the aluminum lake such as Red No. 40 be preferably 0.5 to 30.0 mass %, more preferably 1.0 to 25.0 mass %, with respect to a total amount of the eyeliner composition.

As the colorant other than the aluminum lake such as Red No. 40, any pigment generally used in eyeliners can be used, and examples thereof include: inorganic pigments, such as carbon black, black titanium oxide, yellow iron oxide, black iron oxide, red iron oxide, ultramarine blue, iron blue, chromium oxide, chromium hydroxide, carmine, and shikonin; and organic pigments, for example, barium lake, calcium lake, or zirconium lake pigments of water-soluble dyes such as Red No. 2, Red No. 3, Red No. 102, Red No. 104(1), Red No. 105(1), Red No. 106, Red No. 227, Red No. 230(1), Red No. 230(2), Red No. 231, Red No. 232, Yellow No. 5, Yellow No. 202(1), Yellow No. 202(2), Yellow No. 203, Green No. 3, Green No. 201, Green No. 204, Green No. 205, Blue No. 2, Blue No. 202, Blue No. 205, Orange No. 205, Orange No. 207, and Brown No. 201, as well as Red No. 201, Red No. 202, Red No. 203, Red No. 204, Red No. 205, Red No. 206, Red No. 207, Red No. 208, Red No. 215, Red No. 218, Red No. 219, Red No. 220, Red No. 221, Red No. 223, Red No. 225, Red No. 226, Yellow No. 201, Yellow No. 204, Yellow No. 205, Green No. 202, Blue No. 201, Blue No. 204, Blue No. 404, Orange No. 201, Orange No. 203, Orange No. 204, Orange No. 206, Orange No. 401, Orange No. 402, Orange No. 403, and Violet No. 201. At least one of these colorants can be used.

When any of these colorants is used, from the standpoint of drawn line density, ease of toning, storage stability, and the like, the content of the colorant is 0.1 to 25.0 mass % with respect to a total amount of the eyeliner composition.

As the film-forming agent that can be used specifically, it is desirable to use an aqueous emulsion of preferably an acrylic acid-based polymer.

Examples of the aqueous emulsion of an acrylic acid-based polymer include alkyl acrylate copolymer emulsions, such as YODOSOL GH800F (manufactured by Nouryon Japan K.K., product name, solid concentration: 45 mass %), YODOSOL GH810F (manufactured by Nouryon Japan K.K., product name, solid concentration: 46 mass %), YODOSOL, GH34F (manufactured by Nouryon Japan K.K., product name, solid concentration: 42 mass %), and DAITOSOL 5000SJ (manufactured by Daito Kasei Kogyo Co., Ltd., product name, solid concentration: 50 mass %). For example, COVACRYL MSII (manufactured by Sensient Technologies Corporation, solid content: 57%) that is an alkyl acrylate copolymer emulsion can be used. Examples of an alkyl acrylate-styrene copolymer emulsion include YODOSOL GH4IF (manufactured by Nouryon Japan K.K., product name, solid concentration: 45 mass %), DAITOSOL 5000STY (manufactured by Daito Kasei Kogyo Co., Ltd., product name, solid concentration: 50 mass %), and EMUPOLY CE-119N (sold by Nikko Chemicals Co., Ltd., product name). Examples of an alkyl acrylate-vinyl acetate copolymer emulsion include VINYSOL 2140L (manufactured by Daido Chemical Corporation, product name, solid concentration: 45 mass %).

From the standpoint of water resistance, drawn line adherence, applicability, and the like, it is desired that the content of these resins be 3.0 to 20.0 mass %, preferably 5.0 to 15.0 mass %, with respect to a total amount of the eyeliner composition, in terms of solid content.

The water-soluble organic solvent that can be further added may be, for example, a lower alcohol. The lower alcohol can be preferably used because of its low-temperature stability, drying properties, low irritation, and the like. Specifically, the water-soluble organic solvent may be, for example, at least one (one or a mixture of two or more) of methyl alcohol (methanol), ethyl alcohol (ethanol), n-propyl alcohol, isopropyl alcohol, n-butyl alcohol, sec-butyl alcohol, tert-butyl alcohol, isobutyl alcohol, pentyl alcohol, ethylene glycol, propylene glycol, butylene glycol, and the like.

Particularly, from the standpoint of safety, ease of handling, and the like, it is desirable to use ethyl alcohol (ethanol).

The content of the lower alcohol is preferably 1 to 80 mass %, more preferably 1 to 40 mass %, particularly preferably 5 to 20 mass %, with respect to a total amount of the eyeliner composition.

It is noted here that the pH can be suitably adjusted with a pH modifier or the like until an alkali-soluble-type acrylic resin is dissolved. As the pH modifier, for example, 2-amino-2-methyl-1-propanol, 2-amino-2-methyl-1,3-propanediol, triethanolamine, L-arginine, aqueous ammonia, or sodium hydroxide can be used, and the pH modifier is particularly preferably aqueous ammonia or 2-amino-2-methyl-1-propanol.

The remainder of the eyeliner composition is adjusted with water (e.g., purified water, distilled water, ion-exchanged water, pure water, or tap water).

As for the content of this water, it is desired that a ratio of the water with respect to all solvents (water+water-soluble organic solvent) be preferably 30 to 95 mass % (0.30 to 0.95), more preferably 30 to 80 mass %.

When the ratio of the water with respect to all solvents is lower than 30 mass % (0.30), the stability with time is poor, and the dissolved stability is deteriorated with an addition of a water-soluble component, whereas when the ratio is higher than 95 mass % (0.95), the antibacterial property is highly likely to be poor.

The eyeliner composition configured in the above-described manner contains at least the above-described pigment dispersion for aqueous cosmetic compositions that contains Red No. 40, film-forming agent, water and water-soluble organic solvent, and pH modifier and the like. The eyeliner composition may further contain, as appropriate, other raw materials such as a thickener, a surfactant, a preservative, a UV absorber, an antioxidant, a reduction inhibitor, a chelating agent, an oily component, a fragrance, and an animal/plant extract, within a range that does not impair the effects of the present invention.

Examples of an antimicrobial agent that can be used include parabens, sodium dehydroacetate, phenoxyethanol, and 1,2-hexanediol. The antimicrobial agent in the present invention contains a preservative and, as a paraben serving as the preservative, for example, methyl p-hydroxybenzoate, ethyl p-hydroxybenzoate, propyl p-hydroxybenzoate, butyl p-hydroxybenzoate, or isopropyl p-hydroxybenzoate can be used in an appropriate amount.

From the standpoint of applicability, storage stability, inhibition of pigment precipitation, and the like, examples of a thickener that can be used include: cellulose-based thickeners, such as hydroxyethyl cellulose, hydroxypropylmethyl cellulose, stearoxyhydroxypropylmethyl cellulose, hydroxypropyl guar hydroxypropyltrimonium chloride, and cationized cellulose in which a cationic functional group is added to cellulose; resin-based thickeners, such as polyvinyl alcohol and acrylic acid; clay-based thickeners, such as bentonite; and polysaccharide-based thickeners, such as xanthan gum.

From the standpoint of improving the adherence such as proper viscosity and water resistance and improving the usability and the applicability for use in an applicator, as well as from the standpoint of the discharging properties and the applicability to around the eyes in the case of using the eyeliner composition in an autopen-type applicator, the eyeliner composition desirably has a viscosity at 25° C. (cone plate-type viscometer: 50 rpm; shear rate=192 s) of 1.0 to 200 mPa·s, preferably 1.5 to 40 mPa·s, more preferably 1.5 to 15.0 mPa·s.

The viscosity can be adjusted to be in the above-described range (1.0 to 200 mPa·s) by suitably adjusting the amounts of the above-described respective components to be used, the type and the amount of the above-described thickener to be used preferably, and the like.

By controlling the viscosity of the eyeliner composition to be 1.0 mPa·s or more, liquid leakage from a container or the like is made unlikely to occur, and the stability of a drawn line after application is improved, while by controlling the viscosity to be 200 mPa·s or less, the composition can be discharged in an appropriate liquid amount by an operation of the user.

The eyeliner composition of the present embodiment can be prepared by any conventional method, and an eyeliner composition having a viscosity and a pH in the above-described respective preferred ranges can be produced by blending the above-described pigment dispersion for aqueous cosmetic compositions that contains Red No. 40 with components such as a film-forming agent, water, a water-soluble organic solvent, and an antimicrobial agent in the above-described respective content ranges, and uniformly stirring and mixing the resultant using a suitable kneader or the like.

For the use of the eyeliner composition of the present embodiment that is configured in the above-described manner, a general-purpose cosmetic applicator can be used, and the shape, the structure, and the like of the cosmetic applicator to be used are not particularly limited. Examples of the cosmetic applicator include an applicator equipped with a knocking-type valve device, a hair mascara-type applicator, a tube-type applicator, and an applicator equipped with a piston pressing mechanism.

The resulting aqueous cosmetic composition can be stored in a pen-type applicator having a brush as an application part. For example, the aqueous cosmetic composition can be used by storing (filling) it in a pen-type liquid cosmetic applicator (container) having a brush, a pen core, or the like as an application part.

The liquid cosmetic applicator that can be used is not particularly limited as long as it is, for example, a liquid cosmetic applicator equipped with a brush or a pen core for eyeliners or eyebrows.

One preferred example of such a liquid cosmetic applicator is an applicator which includes a container to be filled with a liquid cosmetic and is equipped with an application body serving as an application means that is constituted by a brush (brush pen) or a pen core for eyeliners or eyebrows, and a rubber, an elastomer, or a resilient closed-cell foam.

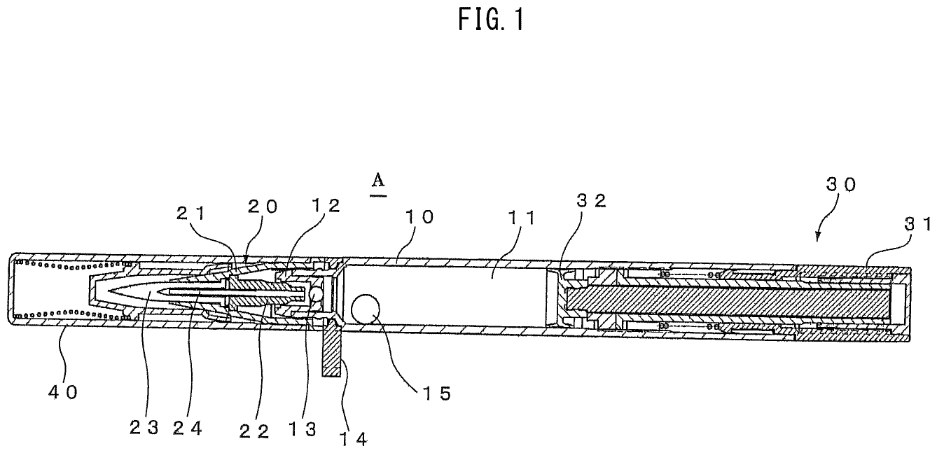

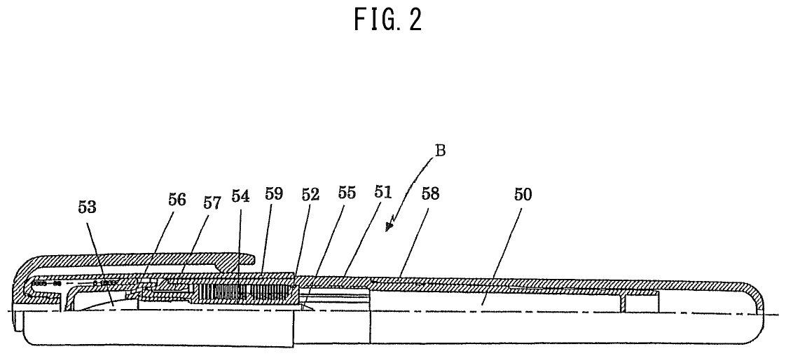

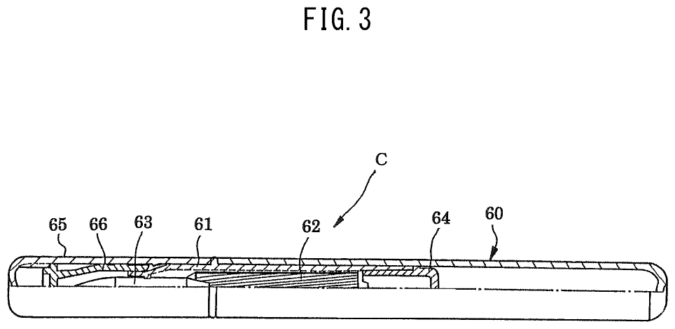

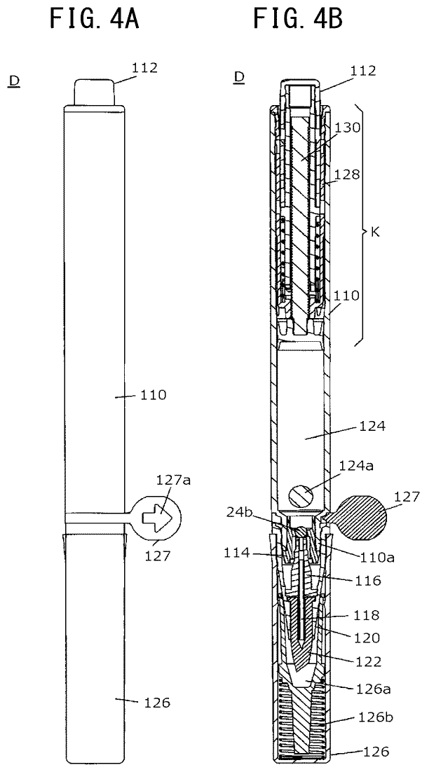

Specifically, it is desirable to use a liquid cosmetic applicator excellent in usability, convenience, and applicability, which has a mechanism of any of the rotary feeding type, the collector type, the cosmetic built-in type, and the knocking type that are illustrated in FIG. 1, FIG. 2, FIG. 3, and FIGS. 4A and 4B, respectively.

As illustrated in FIG. 1, a rotary feeding-type liquid cosmetic applicator A includes an application part 23 formed of a brush (brush pen), which is provided in front of a cosmetic-storing container 11 serving as a reservoir from which the liquid cosmetic of the present invention (hereinafter, simply referred to as “liquid cosmetic”) stored in front of a liquid pressing mechanism 30 is discharged by means of the liquid pressing mechanism 30.

The liquid pressing mechanism 30 is configured such that the liquid cosmetic in the container (reservoir) 11 is drawn out and supplied to the application part 23 by relative rotation of a feeding member 31, which is arranged in a rear-end part of a shaft main body 10, in a circumferential direction with respect to the shaft main body 10.

The liquid pressing mechanism 30 of this applicator includes: the feeding member 31 which is rotatably fitted to the rear end of the shaft main body 10; a driving cylinder which transmits a rotating force of the feeding member 31 applied by a user to a screw rod; a screw body which is fixed to the shaft main body 10 and screwed together with the screw rod; the screw rod with which a piston body 32 is rotatably engaged at a tip; and the piston body 32 which slides inside the reservoir 11 of the shaft main body 10. The liquid pressing mechanism 30 has a structure in which the rotation of the feeding member 31 is transmitted to the screw rod via the driving cylinder, and the resulting rotation of the screw rod causes the screw rod and the piston body 32 to move forward via a female screw of the nut-shaped screw body, whereby the liquid cosmetic is fed from the reservoir 11 to the application part 23.

In the feeding member 31, a cylindrical operating part, which is closed by inserting a crown into the rear end, is rotatably fitted to the rear-end part of the shaft main body 10 and exposed. The driving cylinder is inserted into the feeding member 31 and fixed in a rotational direction, and in this driving cylinder, the screw body is mounted with a fixed rotational direction in a manner to be relatively movable in the axial direction. The crown is a spring member, and biases the feeding member 31, which is a rotating body, to the rear side.

In this applicator, a sealing part, a joint member 22, a front shaft 20, and the application part 23 are attached to the front-end part of the shaft main body 10 by insertion. The liquid cosmetic is stored in the reservoir 11 of the shaft main body 10, and the liquid cosmetic drawn out from the reservoir 11 passes through a flow path inside the joint member and is discharged to the application part 23, enabling to apply the liquid cosmetic. Further, the applicator is configured such that, after its use, a cap 40 can be mounted on the front shaft 20 to cover the application part 23 and the front shaft 20.

In FIG. 1, the symbol 15 represents a stirring ball which stirs the liquid cosmetic in the reservoir 11 by reciprocating motion, and the symbol 13 represents a sealing ball. An inner cap is provided in the cap 40, and this inner cap is biased to the rear by a rear-biasing spring. It is noted here that the stirring ball 15 may be omitted when the liquid cosmetic has good dispersibility as in the present invention.

Further, the symbol 14 represents a stopper in which a ring-shaped part is mounted between the rear end of the front shaft 20 and the front face of a stepped portion of a front-end part 12 of the shaft main body 10 so as to arrange the sealing part, the joint member 22, the front shaft 20, and the application part 23 at a position where the flow path of the liquid cosmetic toward the application part 23 is closed when the applicator is not in use. In this stopper 14, the ring-shaped part is partially cut off, and a knob piece is integrally formed on the opposite side of the cut-off portion. The ring-shaped part can be detached from the rear end of the front shaft 20 and the front-end part 12 of the shaft main body 10 by pulling the knob piece and thereby expanding the diameter of the ring-shaped part from the cut-off portion.

As illustrated in FIG. 1, when the applicator is not in use, the sealing ball 13 is inserted into and tightly seals an inner diameter portion of the sealing part that serves as a sealing ball receptor, preventing the liquid cosmetic from flowing to the side of the application part 23. On the other hand, when the applicator is used, the user pulls the stopper 14 out of the shaft main body 10 and pushes the front shaft 20 to the rear-end side, and this causes a rear-end narrow portion of the joint member 22 to bump into the sealing ball 13, whereby the sealing ball 13 is removed from the inner diameter portion of the sealing part and moves into the reservoir 11, as a result of which the liquid cosmetic in the reservoir 11 flows from the inner diameter portion of the joint member 22 into the liquid flow path of the application part 23 and is supplied from the inside of the liquid flow path to the application part 23, enabling to apply the liquid cosmetic to a target part.

FIG. 2 illustrates a collector-type liquid cosmetic applicator of a direct-feed system in which the liquid cosmetic composition for eye makeup according to the present invention is stored.

In this liquid cosmetic applicator B, for example, as illustrated in FIG. 2, the aqueous cosmetic composition 50 of the present invention is filled into a tank part 51 constituting a shaft body in which the aqueous cosmetic composition 50 is directly stored without being absorbed by inner cotton or the like. The liquid cosmetic applicator B is configured such that a leaflet body (ink retainer, collector member) 52, which temporarily retains the liquid cosmetic 50 pushed out from the tank part 51 in the event of expansion of the air inside the tank part 51 due to a temperature increase or the like so as to prevent dripping of the liquid cosmetic 50 from a pen tip or an air hole, is built-in in the front portion of the tank part 51, and a brush-type pen tip (brush) 53 serving as an application body is provided on the tip of the collector member 52. As the brush-type pen tip (brush) 53, for example, a brush having an irregular cross-sectional shape other than a circular shape or a square shape.

The aqueous cosmetic composition 50 is delivered from the tank part 51 to the pen tip 53 serving as an application part via a relay core 55 in which an application liquid flow path 54 attached to a center hole of the collector member 52 is provided.

In FIG. 2, the symbols 56 and 57 each represent a holder member, the symbol 58 represents a rear shaft body fixed to the rear of the tank part 51, and the symbol 59 represents a cap having an inner cap. The aqueous cosmetic composition may also be delivered by arranging a rear portion of the pen tip 53 directly in the tank part 51, without interposing the relay core 55 therebetween.

The liquid cosmetic applicator B of this form is used by removing the cap 59 in the same manner as the above-described liquid cosmetic applicator A.

FIG. 3 illustrates an inner cotton-type liquid cosmetic applicator C in which the aqueous cosmetic composition for eye makeup according to the present invention is used.

The liquid cosmetic applicator C of this form has, for example, as illustrated in FIG. 3, a structure in which an inner shaft 61 is provided inside an applicator main body 60; an impregnation body 62, which is formed of a porous material impregnated with the aqueous cosmetic composition of the present invention, is housed in the inner shaft 61; an application body 63, which is formed of a brush having an irregular cross-sectional shape for application of the liquid cosmetic, is provided on the tip side of the impregnation body 62; and a tail plug 64 is fixed to the rear end of the inner shaft 61. The symbol 65 represents a cap having an inner cap part 66. The liquid cosmetic applicator C of this form is used by removing the cap 65.

FIG. 4 illustrates a direct-feed knocking-type liquid cosmetic applicator D in which the aqueous cosmetic composition for eye makeup according to the present invention is used.

As illustrated in FIGS. 4(a) and (b), the liquid cosmetic applicator D is a container from which the liquid cosmetic in a reservoir 124 can be drawn out by pressing a crown 112, which is arranged in a rear-end part of a shaft main body 110, forward in the axial direction. More specifically, the liquid cosmetic applicator D includes: a knocking mechanism K which converts a pressing force of the crown 112 generated by a knocking operation of a user into a rotational force; a screw body 128 which is fixed to the shaft main body 110; and a screw rod 130 which is screwed with the screw body 128. By rotating the screw rod 130 with the rotational force converted by the knocking mechanism K, the screw rod 130 is moved forward via the screw body 128, and the content is thereby drawn out.

In the above-described cosmetic applicator D, a joint 114, a pipe joint 116, a pipe 118, a front shaft 120, and a brush head 122 are attached to a front-end part 110a of the shaft main body 110, and the content (aqueous cosmetic composition) drawn out from the reservoir 124 in the shaft main body 110 passes through the pipe 118 and is discharged to the brush head 122. Further, the cosmetic applicator D is configured such that a cap 126 can be fitted thereto after use.

It is noted here that the symbol 124a represents a stirring ball which stirs the content in the reservoir 124 by reciprocating motion; the symbol 126a represents an inner cap; the symbol 126b represents a spring for rear-biasing the inner cap; and the symbol 127 represents a stopper that blocks the flow of the content to the downstream of the pipe 118 when the cosmetic applicator D is not in use. In a rear-end part of the pipe 118, when the cosmetic applicator D is not in use, a sealing ball 124b tightly adheres to an inner diameter portion of the joint 114 such that the content does not flow into the pipe 118. On the other hand, when the cosmetic applicator D is in use, the sealing ball 124b is detached from the inner diameter portion of the joint 114 by pulling out the stopper 127 from the shaft main body 110 and pushing the front shaft 120 to the rear-end side, as a result of which the content flows into the pipe 118 and can be applied.

### EXAMPLES

The present invention will now be described in more detail by way of Examples and Comparative Examples; however, the present invention is not limited by the below-described Examples and the like.

### Production Examples 1 to 13 and Comparative Production Examples 1 to 10: Preparation of Pigment Dispersions for Aqueous Cosmetic Compositionss

In the same proportion as in the respective blending formulations shown in Tables 1 and 2 below, an aluminum lake such as Red No. 40, and a dispersant selected from polyorganic acids and salts or nonionic surfactants were dispersed in water and a water-soluble organic solvent using a bead mill to prepare pigment dispersions for cosmetics.

### Examples 1 to 13 (Table 1) and Comparative Examples 1 to 10 (Table 2): Preparation of Eyeliner Compositions

A film-forming agent and the like were further added to each of the pigment dispersions for aqueous cosmetic compositions that were obtained in Production Examples 1 to 13 and Comparative Production Examples 1 to 10, and the resultants were each mixed and stirred to achieve the respective blending formulations shown in Tables 1 and 2 below, whereby eyeliner compositions were prepared.

The results of evaluating these eyeliner compositions are shown in Tables 1 and 2 below.

### [Measurement of Viscosity of Eyeliner Compositions]

For each of the eyeliner compositions of Examples 1 to 13 and Comparative Examples 1 to 10 that were obtained by the above-described method, the viscosity was measured at 25° C. using a cone plate-type viscometer (TVE-25, manufactured by Toki Sangyo Co., Ltd.) under a shear rate of 192 s⁻¹.

### (Measurement of Particle Size of Eyeliner Compositions)

For each of the above-obtained pigment dispersions for aqueous cosmetic compositions, the average particle size was measured at 25° C. using a particle size distribution analyzer [FPAR-1000 (manufactured by Otsuka Electronics Co., Ltd.)].

### (Evaluation Method of Applicability)

The above-obtained eyeliner compositions were each filled into the above-described applicator B (collector-type eyeliner container) and applied to the skin of a monitor, and the applicability was evaluated based on the following evaluation criteria.
A: The cosmetic was discharged and applied with no problem.
B: The cosmetic was discharged and applied to the skin; however, a drawn line was partially blurry.
C: The cosmetic was not discharged normally from the container, and could not be applied to the skin.
   (Color Development after Application)

In the same manner as in the above-described evaluation of the applicability, the eyeliner compositions were each filled into the applicator B and applied to the skin of 10 monitors, and the color development was evaluated based on the following evaluation criteria.
A: All of the 10 monitors felt that the color development was excellent.
B: At least 7 of the 10 monitors felt that the color development was good.
C: At least 3 of the 10 monitors felt that the color development was good.
D: None of the monitors felt that the color development was good.

### (Adherence of Drawn Line)

In the same manner as in the above-described evaluation of the applicability, the eyeliner compositions were each filled into the applicator B and applied to the skin of 10 monitors. After a drawn line was dried, the applied surface was rubbed with the ball of a finger, and the adherence was evaluated based on the following evaluation criteria.
A: The drawn line had no change even after being rubbed 10 times with the ball of a fiber of each monitor who performed the application.
B: As a result of rubbing the drawn line 10 times with the ball of a finger of each monitor who performed the application, three of the 10 monitors recognized a slight change in the drawn line.
C: As a result of rubbing the drawn line 10 times with the ball of a finger of each monitor who performed the application, 7 of the 10 monitors recognized a change in the drawn line.
D: As a result of rubbing the drawn line 10 times with the ball of a finger of each monitor who performed the application, all of the monitors recognized a change in the drawn line, or the drawn line was erased.

### (Water Resistance)

In the same manner as in the above-described evaluation of the applicability, the above-obtained eyeliner compositions were each filled into the applicator B and applied to the skin. After a drawn line was dried, the above-described adherence test of the applied surface was conducted in running water, and the water resistance was evaluated based on the following evaluation criteria.
A: The drawn line had no change even after being rubbed 10 times with the ball of a fiber of each monitor who performed the application.
B: As a result of rubbing the drawn line 10 times with the ball of a finger of each monitor who performed the application, 3 of the 10 monitors recognized a slight change in the drawn line.
C: As a result of rubbing the drawn line 10 times with the ball of a finger of each monitor who performed the application, 7 of the 10 monitors recognized a change in the drawn line.
D: As a result of rubbing the drawn line 10 times with the ball of a finger of each monitor who performed the application, all of the monitors recognized a change in the drawn line, or the drawn line was erased.

### (Makeup Removability (Non-Staining Property on Skin)

In the same manner as in the above-described evaluation of the applicability, the above-obtained eyeliner compositions were each filled into the applicator B and applied to the skin. After a lapse of at least 8 hours, the makeup on the applied surface was removed with a makeup remover (BIORE (registered trademark) Wipe-Only Cotton Makeup Remover; manufactured by Kao Corporation), and the makeup removability (staining property) was evaluated based on the following evaluation criteria.
A: A drawn line was not recognizable after the makeup removal.
B: A drawn line remained even after the makeup removal (pigmentation occurred).

TABLE 1

| | | Exam- | Exam- | Exam- | Exam- | Exam- | Exam- | Exam- |
|---|---|---|---|---|---|---|---|---|
| | | ple 1 | ple 2 | ple 3 | ple 4 | ple 5 | ple 6 | ple 7 |
| Coloring | Red No. 40 lake | 9 | 9 | 15 | 28 | 28 | 9 | 9 |
| material | Blue No. 1 lake | | | | | | | |
| | Yellow No. 4 lake | | | | | | | |
| | Red No. 40 | | | | | | | |
| | Blue No. 1 | | | | | | | |
| | Yellow No. 4 | | | | | | | |
| Emulsion resin | Alkyl acrylate | 8.4 | 8.4 | 8.4 | 8.4 | 8.4 | 8.4 | 8.4 |
| (adhesive) | copolymer emulsion | | | | | | | |
| Polyorganic | Sodium polyacrylate | 0.09 | 0.9 | 6 | 11.2 | 14 | | |
| acid (salt) | Sodium polyaspartate | | | | | | 3.6 | |
| Nonionic | (Styrene/acrylate) | | | | | | | 3.6 |
| polymer | copolymer | | | | | | | |
| Nonionic | POE(20) cetyl ether | | | | | | | |
| surfactant | | | | | | | | |
| Cationic | PPG-1/PEG-1 | | | | | | | |
| surfactant | stearamine | | | | | | | |
| Preservative | | 4.06 | 4.06 | 4.06 | 4.06 | 4.06 | 4.06 | 4.24 |
| Aqueous solvent | | remainder | remainder | remainder | remainder | remainder | remainder | remainder |
| (including | | | | | | | | |
| purified | | | | | | | | |
| water) | | | | | | | | |
| Physical | Viscosity, mPa · s: | 4.4 | 5.6 | 12.5 | 17.5 | 19.8 | 18.1 | 9.2 |
| properties | 50 rpm | | | | | | | |
| of cosmetic | (shear rate: 192/s) | | | | | | | |
| | Particle size | 251 | 260 | 291 | 321 | 307 | 284 | 281 |
| Evaluation | Applicability | A | A | A | A | A | A | A |
| items | Color development | B | B | A | A | A | B | B |
| | Adherence | A | A | A | A | A | A | A |
| | Water resistance | A | B | B | B | B | B | A |
| | Makeup removability | A | A | A | A | A | A | A |
| | (staining property) | | | | | | | |

| | | Exam- | Exam- | Exam- | Exam- | Exam- | Exam- |
|---|---|---|---|---|---|---|---|
| | | ple 8 | ple 9 | ple 10 | ple 11 | ple 12 | ple 13 |
| Coloring | Red No. 40 lake | 9 | 15 | | | 8 | 5 |
| material | Blue No. 1 lake | | | 9 | | | |
| | Yellow No. 4 lake | | | | 9 | | |
| | Red No. 40 | | | | | | |
| | Blue No. 1 | | | | | | |
| | Yellow No. 4 | | | | | | |
| Emulsion resin | Alkyl acrylate | 8.4 | 3 | 8.4 | 8.4 | 25.2 | 3 |
| (adhesive) | copolymer emulsion | | | | | | |
| Polyorganic | Sodium polyacrylate | | 6 | 0.09 | 0.09 | 0.8 | 1 |
| acid (salt) | Sodium polyaspartate | | | | | | |
| Nonionic | (Styrene/acrylate) | | | | | | |
| polymer | copolymer | | | | | | |
| Nonionic | POE(20) cetyl ether | 4.5 | | | | | |
| surfactant | | | | | | | |
| Cationic | PPG-1/PEG-1 | | | | | | |
| surfactant | stearamine | | | | | | |
| Preservative | | 4.06 | 4.06 | 4.06 | 4.06 | 4.18 | 4.06 |
| Aqueous solvent | | remainder | remainder | remainder | remainder | remainder | remainder |
| (including | | | | | | | |
| purified | | | | | | | |
| water) | | | | | | | |
| Physical | Viscosity, mPa · s: | 9 | 7.5 | 4.2 | 4.7 | 14.1 | 2.7 |
| properties | 50 rpm | | | | | | |
| of cosmetic | (shear rate: 192/s) | | | | | | |
| | Particle size | 158 | 287 | 281 | 255 | 257 | 284 |
| Evaluation | Applicability | A | A | A | A | A | A |
| items | Color development | B | A | B | B | B | B |
| | Adherence | A | B | A | A | A | B |
| | Water resistance | B | B | A | A | A | B |
| | Makeup removability | A | A | A | A | A | A |
| | (staining property) | | | | | | |

TABLE 2

| | | Comparative | Comparative | Comparative | Comparative | Comparative | Comparative |
|---|---|---|---|---|---|---|---|
| | | Example 1 | Example 2 | Example 3 | Example 4 | Example 5 | Example 6 |
| Coloring | Red No. 40 lake | 9 | 35 | 9 | 15 | | |
| material | Blue No. 1 lake | | | | | | |
| | Yellow No. 4 lake | | | | | | |
| | Red No. 40 | | | | | 1 | |
| | Blue No. 1 | | | | | | 1 |
| | Yellow No. 4 | | | | | | |
| Emulsion | Alkyl acrylate | 8.4 | 8.4 | 8.4 | 1.5 | 8.4 | 8.4 |
| resin | copolymer | | | | | | |
| (adhesive) | emulsion | | | | | | |
| Polyorganic | Sodium | | 17.5 | 0.03 | 6 | | |
| acid (salt) | polyacrylate | | | | | | |
| | Sodium | | | | | | |
| | polyaspartate | | | | | | |
| Nonionic | (Styrene/ | | | | | | |
| polymer | acrylate) | | | | | | |
| | copolymer | | | | | | |
| Nonionic | POE(20) cetyl | | | | | | |
| surfactant | ether | | | | | | |
| Cationic | PPG-1/PEG-1 | | | | | | |
| surfactant | stearamine | | | | | | |
| Preservative | | 4.06 | 4.06 | 4.06 | 4.06 | 4.06 | 4.06 |
| Aqueous solvent | | remainder | | remainder | remainder | remainder | remainder |
| (including | | | | | | | |
| purified water) | | | | | | | |
| Physical | Viscosity, mPa · s: | not measurable | 34.7 | 3.1 | 5.4 | 3.2 | 3 |
| properties of | 50 rpm | (gelation) | | | | | |
| cosmetic | (shear rate: 192/s) | | | | | | |
| | Particle size | not measurable | 387 | 275 | 288 | Water-soluble dye: no particle size | |
| | | (gelation) | | | | | |
| Evaluation | Applicability | C | C | A | A | A | A |
| items | Color development | not discharged: | not discharged: | C | A | B | B |
| | | not evaluated | not evaluated | | | | |
| | Adherence | not discharged: | not discharged: | C | D | B | B |
| | | not evaluated | not evaluated | | | | |
| | Water resistance | not discharged: | not discharged: | B | C | C | C |
| | | not evaluated | not evaluated | | | | |
| | Makeup | not discharged: | not discharged: | A | A | B | B |
| | removability | not evaluated | not evaluated | | | | |
| | (staining property) | | | | | | |

| | | Comparative | Comparative | Comparative | Comparative |
|---|---|---|---|---|---|
| | | Example 7 | Example 8 | Example 9 | Example 10 |
| Coloring | Red No. 40 lake | | 9 | 9 | 5 |
| material | Blue No. 1 lake | | | | |
| | Yellow No. 4 lake | | | | |
| | Red No. 40 | | | | |
| | Blue No. 1 | | | | |
| | Yellow No. 4 | 1 | | | |
| Emulsion | Alkyl acrylate | 8.4 | 35 | 8.4 | 1 |
| resin | copolymer | | | | |
| (adhesive) | emulsion | | | | |
| Polyorganic | Sodium | | 0.9 | | 0.05 |
| acid (salt) | polyacrylate | | | | |
| | Sodium | | | | |
| | polyaspartate | | | | |
| Nonionic | (Styrene/ | | | | |
| polymer | acrylate) | | | | |
| | copolymer | | | | |
| Nonionic | POE(20) cetyl | | | | |
| surfactant | ether | | | | |
| Cationic | PPG-1/PEG-1 | | | 0.9 | |
| surfactant | stearamine | | | | |
| Preservative | | 4.06 | 4.52 | 4.06 | 4.06 |
| Aqueous solvent | | remainder | remainder | remainder | remainder |
| (including | | | | | |
| purified water) | | | | | |
| Physical | Viscosity, mPa · s: | 3.1 | 27.8 | not measurable | 1.3 |
| properties of | 50 rpm | | | (gelation) | |
| cosmetic | (shear rate: 192/s) | | | | |
| | Particle size | Water-soluble dye: | 374 | not measurable | 254 |
| | | no particle size | | (gelation) | |
| Evaluation | Applicability | A | C | C | A |
| items | Color development | B | not discharged: | not discharged: | B |
| | | | not evaluated | not evaluated | |
| | Adherence | B | not discharged: | not discharged: | D |
| | | | not evaluated | not evaluated | |
| | Water resistance | C | not discharged: | not discharged: | D |
| | | | not evaluated | not evaluated | |
| | Makeup | B | not discharged: | not discharged: | A |
| | removability | | not evaluated | not evaluated | |
| | (staining property) | | | | |

As apparent from the results shown in Tables 1 and 2, the eyeliner compositions of Examples 1 to 13 exhibited good discharge and applicability when used in the collector-type applicator B which was provided with a relay core and had a brush as the application part, and were confirmed to be superior to the eyeliner compositions of Comparative Examples 1 to 10 in all of the color development, the adherence, the water resistance, and the makeup removability.

### INDUSTRIAL APPLICABILITY

The following can be obtained: a pigment dispersion for aqueous cosmetic compositions, which is excellent in the dispersibility of aluminum lakes that are pigments, such as Red No. 40; and an aqueous cosmetic composition using the same, which is suitable for eyeliner compositions and the like.

### REFERENCE SIGNS LIST

10, 60, 110: shaft main body
11: reservoir
12: front-end part of shaft main body
13, 124b: sealing ball
14, 127: stopper
15, 124a: stirring ball
20, 120: front shaft
21: front shaft main body
23, 53, 63, 122: brush (application part)
24, 54, 116: relay core, pipe
51: tank part
52: collector member
61: inner shaft
62: impregnation body (inner cotton)
K: knocking mechanism

1. A pigment dispersion for aqueous cosmetic compositions, the pigment dispersion comprising, at least:
an aluminum lake;
at least one dispersant selected from polyorganic acids and salts thereof, and nonionic surfactants; and
water and a water-soluble organic solvent.

2. The pigment dispersion for aqueous cosmetic compositions, wherein the content of the dispersant is 0.05 to 17.00 mass % with respect to a total amount of the pigment dispersion for aqueous cosmetic compositions.

3. An aqueous cosmetic composition, comprising, at least:
an aluminum lake at a ratio of 0.00025 to 30.00 mass %;
0.05 to 15.00 mass % of a dispersant selected from polyorganic acids and salts thereof, and nonionic surfactants;
at least 2.50 to 30.00 mass % of a film-forming agent; and
water and a water-soluble organic solvent.

4. The aqueous cosmetic composition according to claim 3, having a viscosity of 1.5 mPa·s to 20.0 mPa·s at 25° C. and a shear rate of 192 s⁻¹.
